(19) United States
(12) Patent Application Publication
(10) Pub. No.: US 2026/0028567 A1
(43) Pub. Date: Jan. 29, 2026

(54) MICROBIOME PROPAGATION SYSTEM AND METHOD

(71) Applicant: Purdue Research Foundation, West Lafayette, IN (US)

(72) Inventors: Roland Conrad Wilhelm, Lafayette, IN (US); Jeffrey P. Youngblood, Crawfordsville, IN (US); Arval Viji Elango, West Lafayette, IN (US); Mallory Luse, Franklin, IN (US); Yirou Wang, West Lafayette, IN (US)

(21) Appl. No.: 19/278,536

(22) Filed: Jul. 23, 2025

Related U.S. Application Data
(60) Provisional application No. 63/675,038, filed on Jul. 24, 2024.

(51) Int. Cl. C12M 1/00; C12M 1/36

(52) U.S. Cl. CPC C12M 23/20; C12M 23/02; C12M 41/48

(57) ABSTRACT

The system of the present disclosure includes a robot, a magnet coupled to the robot, and a metallic structure. The metallic structure may be substantially spherically shaped. In a specific example, the metallic structure may include a stainless-steel material. The metallic structure may be coated with a material that is suitable for biofilm formation. In a specific example, the magnet may be coated with polyvinyl chloride (PVC). Desirably, the PVC coating may enable biofilm formation without interrupting the magnetic qualities of the metallic structure. Advantageously, the robot may adjust a position of the metallic structure to enable research of microbiome propagation with solid substrates autonomously or semi-autonomously. For instance, this may at least partially automate the analysis of biofilm growth on PVC surfaces, which may help develop safer hydroponic and home water systems.

### CROSS REFERENCE TO RELATED APPLICATIONS

This application claims the benefit of U.S. Provisional Application No. 63/675,038 filed Jul. 24, 2024, the entirety of which is hereby incorporated by reference.

### FIELD

The disclosure generally relates to robotic systems and, more particularly, to robotic systems that analyze microbiome propagation.

### INTRODUCTION

This section provides background information related to the present disclosure which is not necessarily prior art.

The study of microbes requires high throughput experimental platforms to robustly characterize the community assembly, biochemical function, and ecological interactions among microorganisms. One way to achieve sufficient scale may be to use liquid handling robots which offer efficient and reproducible programmable experiments. However, many microbes grow in colonies on solid surfaces, known as biofilms, which are ubiquitous in ‘built environments,’ like hydroponics facilities. While liquid handling robots are increasingly common research tools, they have yet to be adapted to handle solid materials for research related to biofilms formation and function.

Accordingly, there is a continuing need for a robotic system that may handle solid materials for the study of microbiome propagation.

### SUMMARY

In concordance with the instant disclosure, a robotic system that may handle solid materials for the study of microbiome propagation, has surprisingly been discovered.

The system of the present disclosure may include a robot, a magnet coupled to the robot, and a metallic structure. The metallic structure may be substantially spherically shaped. In a specific example, the metallic structure may include a stainless-steel material. The metallic structure may be coated with a material that is suitable for biofilm formation. In a specific example, the magnet may be coated with polyvinyl chloride (PVC). Desirably, the PVC coating may enable biofilm formation without interrupting the magnetic qualities of the metallic structure. Advantageously, the robot may adjust a position of the metallic structure to enable research of microbiome propagation with solid substrates autonomously or semi-autonomously. For instance, this may at least partially automate the analysis of biofilm growth on PVC surfaces, which may help develop safer hydroponic and home water systems.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1A a front perspective view of the system of the present disclosure, further depicting the robot controlling a position of a coated metallic structure, according to one embodiment of the present disclosure;

FIG. 1B is an enlarged view of call-out A taken in FIG. 1A, further depicting the robot controlling the position of the coated metallic structure via a magnet, according to one embodiment of the present disclosure;

FIG. 2 is a front perspective view of the system of the present disclosure, further depicting the robot having a plurality of pipette channels that may pick up and/or reposition one or more coated metallic structures, according to one embodiment of the present disclosure;

FIG. 3 is a cross-sectioned front elevational view of the coated metallic structure having a spherical metallic material coated with a first layer and a second layer, according to one embodiment of the present disclosure;

FIG. 4 is a line graph illustrating a DSC analysis of a PVC pipe sample, according to one embodiment of the present disclosure;

FIG. 5 is a line graph illustrating a DSC analysis of PVC powder, according to one embodiment of the present disclosure;

FIG. 6 is a line graph illustrating a DSC analysis of PVC powder after it was dissolved in DMF, according to one embodiment of the present disclosure;

FIG. 7 is a plot diagram comparing the cell counts (per cm²) and the DNA Yield (per cm²) for between the state of the art and the system of the present disclosure in various jar types; and

FIG. 8 is a flowchart of a method for manufacturing the system of the present disclosure, according to one embodiment of the present disclosure.

### DETAILED DESCRIPTION

The following description of technology is merely exemplary in nature of the subject matter, manufacture, and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping, or distinct) subsume all possible combinations of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the FIG. is turned over, elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

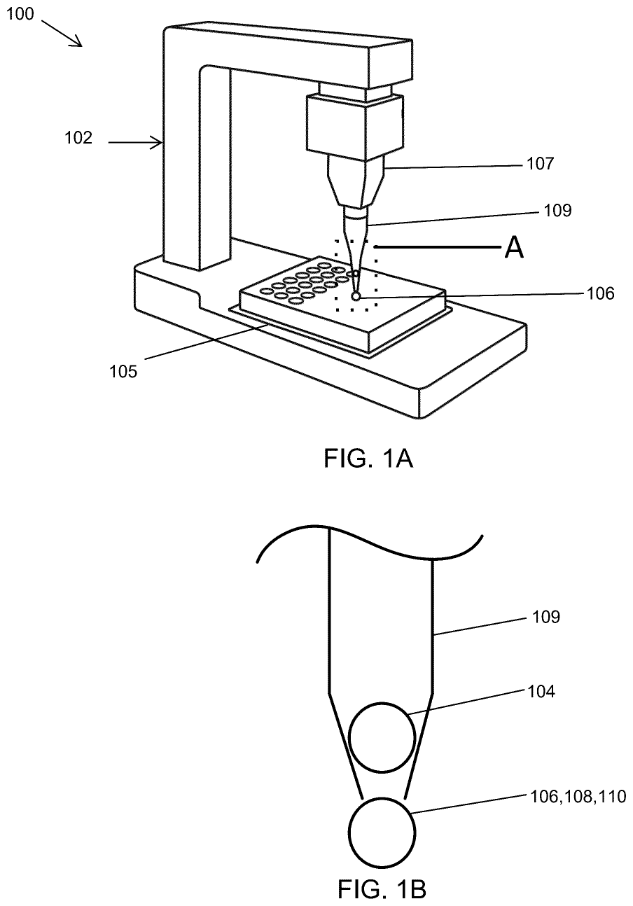

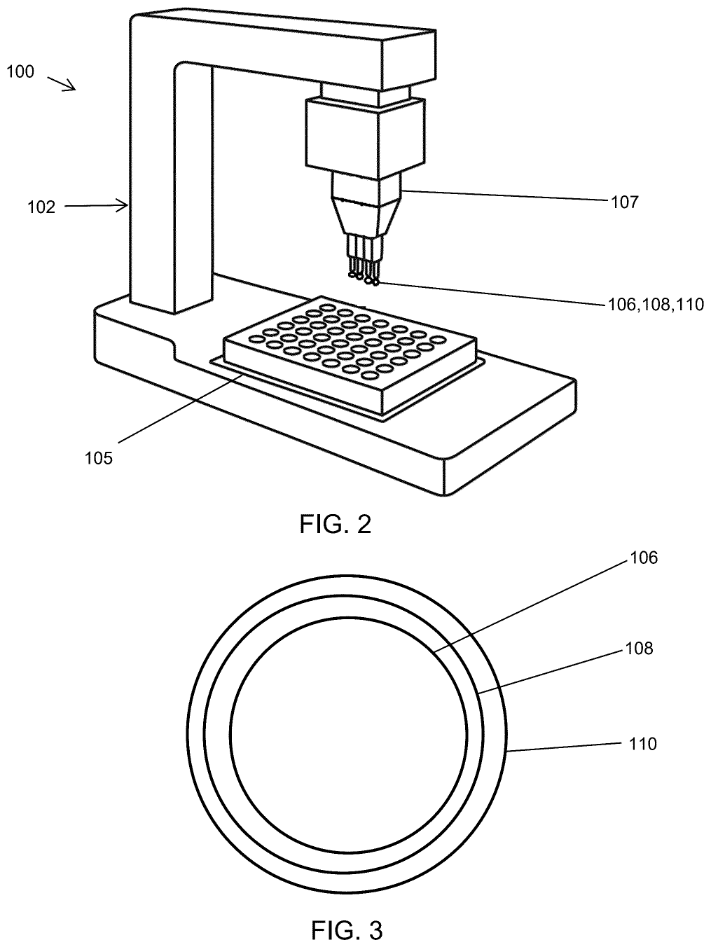

As shown in FIGS. 1A-1B, the system 100 of the present disclosure may include a robot 102, a magnet 104, 105 coupled to the robot 102, and a metallic structure 106. The robot 102 may include a liquid handling robot 102 that is configured to handle solid materials for studying biofilms. The metallic structure 106 may be substantially spherically shaped. In a specific example, the metallic structure 106 may include a stainless-steel material. In a more specific example, the metallic structure 106 may be between around one millimeter to around three millimeters. In certain circumstances, the robot 102 may be configured to pick up and reposition a plurality of metallic structures 106. For instance, as shown in FIG. 2, the robot 102 may include a plurality of pipette channels that may each pick up a metallic structure 106 simultaneously. As shown in FIG. 3, the metallic structure 106 may include a coating 108,110 of a material that is suitable for biofilm formation. In a specific example, the metallic structure 106 may be coated with polyvinyl chloride (PVC) coating 108,110. In a more specific example, the PVC coating 108,110 may include a first coating 108 and a second coating 110. Desirably, the PVC coating 108, 110 may enable biofilm formation without interrupting the magnetic qualities of the metallic structure 106. In certain circumstances, the magnet 104, 105 may include a plurality of magnets, such as a first magnet 104 and a second magnet 105, as shown in FIGS. 1A and 2. Advantageously, the robot 102 may adjust and/or control a position of the metallic structure 106 via the first magnet 104 to enable research of microbiome propagation with solid substrates autonomously or semi-autonomously. In a specific example, a standard pipette tip may be modified by reducing the pipette tip length below the first magnet 104, which may also increase the pipette tip bore size. This may ensure the metallic structure 106 is well seated centrally and securely in a cavity of the modified pipette tip without contacting the first magnet 104 inside the pipette tip. In another specific example, the second magnet 105 may be larger and/or provide a stronger magnetic force compared to the first magnet 104. The robot 102 may also control the second magnet 105 for the purposes of selectively detaching and/or manipulating the metallic structure(s) 106. Provided as a non-limiting example, the second magnet 105 may be selectively movable and/or selectively engaged by the robot 102. For instance, the second magnet 105 may include a switchable electromagnet that may be stronger than the first magnet 104. The robot 102 may also control the functioning of the second magnet 105. Provided as a non-limiting example, the robot 102, when required to deposit the metallic structure 106, may activate or move the second magnet 105 into an activated position. This may exert a stronger attraction on metallic structure 106 than the first magnet 104, allowing the metallic structure 106 to be now deposited into a well plate. Then, the PVC-coated metallic structure 106 may be utilized for downstream processing after being deposited into the well-plate. This may at least partially automate the analysis of biofilm growth on PVC surfaces, which may help develop safer hydroponic and home water systems.

In certain circumstances, the system 100 of the present disclosure may be utilized specifically for biosurveillance. For instance, the metallic structure(s) 106 may be deposited into a hydroponic system and/or a water system. Afterwards, the first magnet 104 on the robot 102 may retrieve the metallic structure(s) 106 from the hydroponic system and/or the water system. In a specific example, the metallic structure(s) 106 may be analyzed for biofilm growth, detect pathogens/diseases, and ensuring the health and productivity of the system. Then, the second magnet 105 may be engaged to redistribute the metallic structure(s) 106 into the hydroponic system and/or the water system. Alternatively, the second magnet 105 may be engaged to position the metallic structure(s) 106 in an area to the cleaned and reused. One skilled in the art may select other suitable ways to use the system 100 of the present disclosure for biosurveillance, within the scope of the present disclosure.

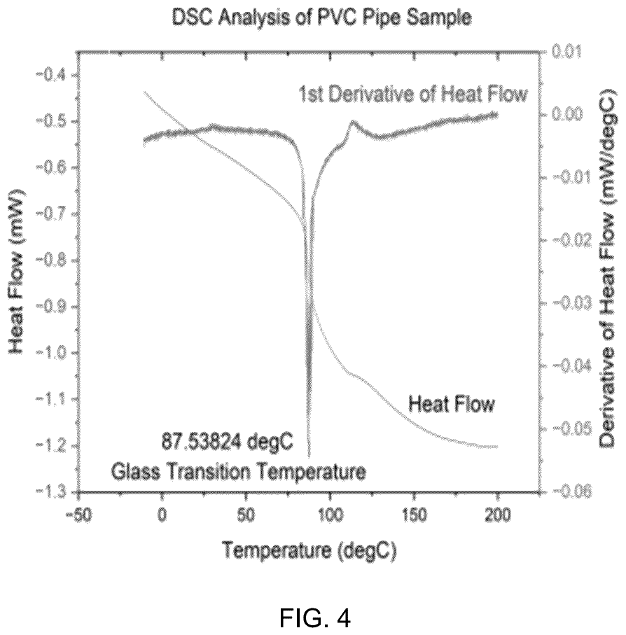

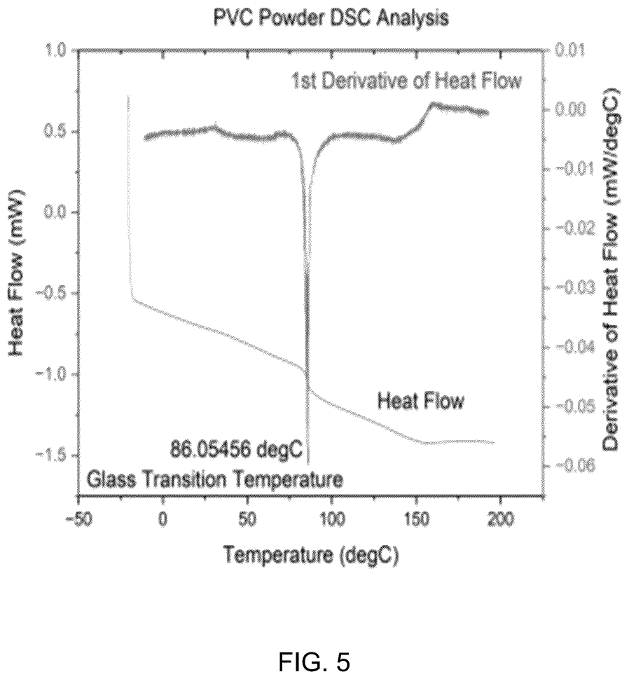

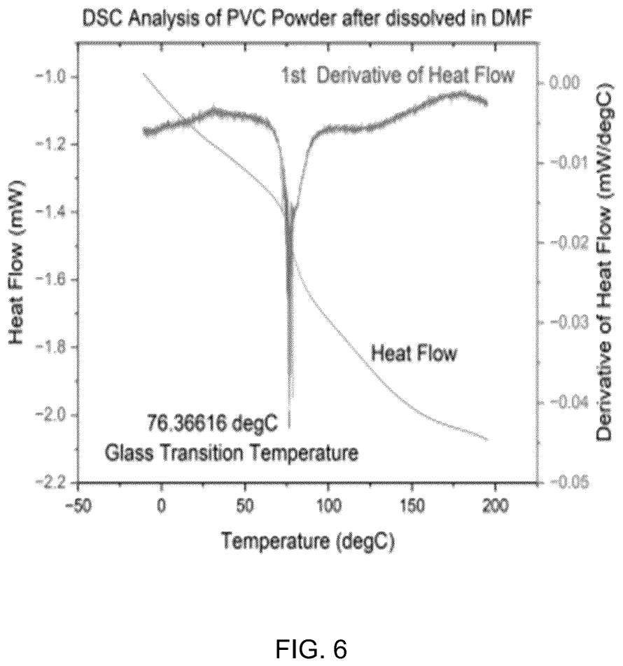

In certain circumstances, different forms of PVC may be utilized for the PVC coating 108,110. For instance, DSC analyses were conducted on PVC pipe, PVC powder, and PVC powder after it was dissolved in DMF, as shown in FIGS. 4-6, respectively. One skilled in the art may select other suitable sources of PVC for the PVC coating 108,110, within the scope of the present disclosure.

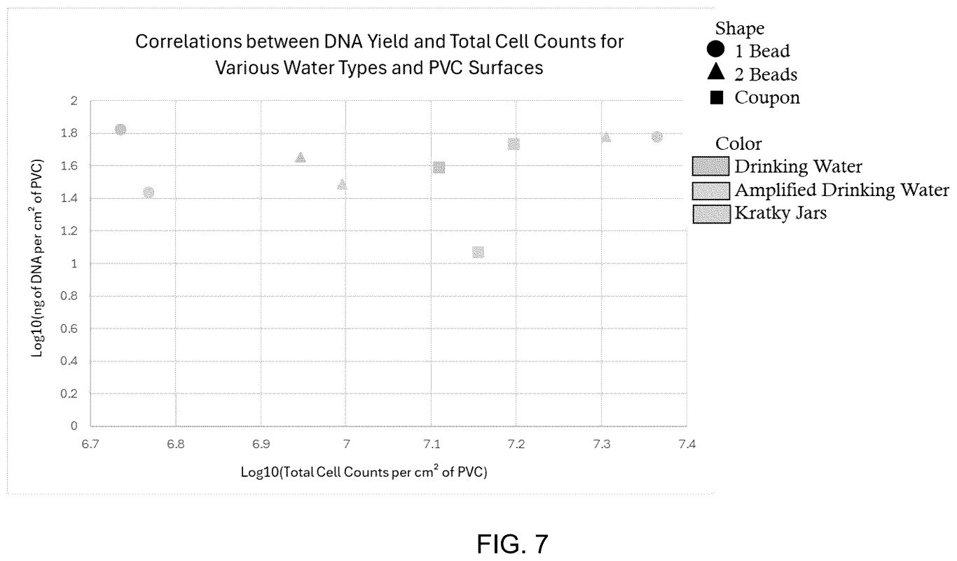

As shown in FIG. 7, experimental tests were conducted comparing the cell counts (per cm²) and the DNA Yield (per cm²) for between the state of the art, termed as “Coupon” and the system of the present disclosure, termed as “1 Bead” and “2 Beads” in various jar types. Overall, the beads of the present disclosure advantageously got equal DNA yields (per cm²) as the coupons across all jar types, even though the beads of the present disclosure have slightly lower biomass. Without being bound to any particular theory, it is believed this advantage stems from a greater compatibility with standard methods for microbiome analysis.

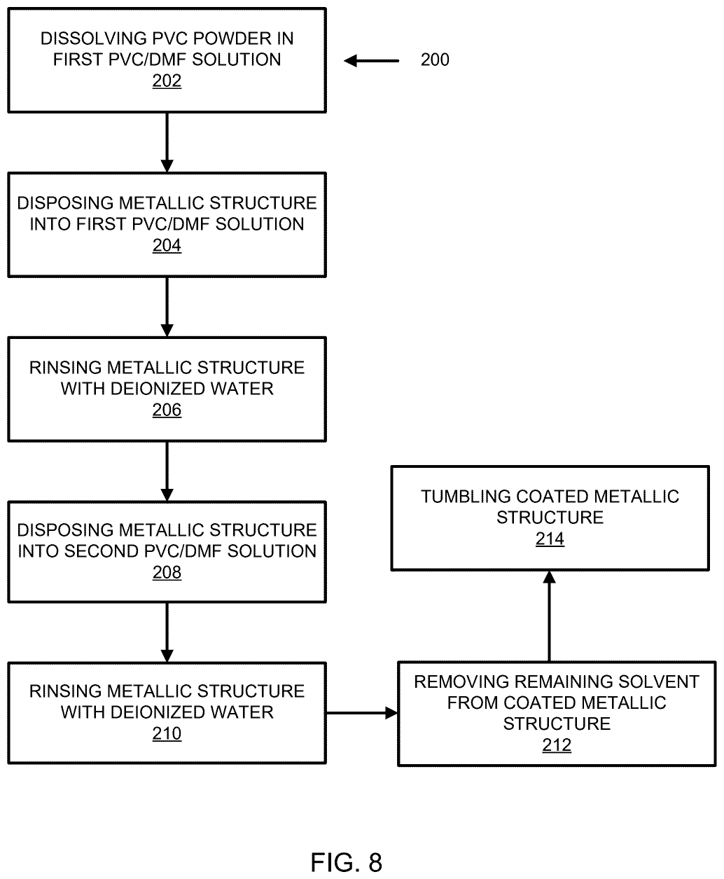

In certain circumstances, the system 100 of the present disclosure may be manufactured in various ways. For instance, as shown in FIG. 8, the system 100 of the present disclosure may be manufactured according to a method 200. The method 200 may include dissolving PVC powder in a solvent. Provided as a non-limiting example, the solvent may include dimethylformamide (DMF), thus providing a first PVC/DMF solution. One skilled in the art may select other suitable solvents, within the scope of the present disclosure. Next, the metallic structure 106 may be dipped in the first PVC/DMF solution. The coated metallic structure 106 may be rinsed with deionized water. In certain circumstances, the metallic structure 106 may be coated with a second PVC/DMF solution and thereafter rinsed with deionized water. The first PVC/DMF solution and the second PVC/DMF solution may include the same or different ratios of PVC and DMF in their solutions. For instance, the first PVC/DMF solution may include around 8% by weight PVC and the second PVC/DMF solution may include around 6% by weight PVC. The remaining solvent from the first PVC/DMF solution and/or the second PVC/DMF solution may be removed from the metallic structure 106. For instance, the metallic structure 106 may be exposed to a vacuum oven to remove the remaining solvent. One skilled in the art may select other suitable ways for manufacturing the system 100 of the present disclosure.

Provided as a non-limiting example, the system 100 of the present disclosure was compared against PVC used in hydroponics growing troughs to further hydroponics microbiome research. The system 100 of the present disclosure included a metallic structure 106 provided as polyvinyl chloride (PVC) coated stainless steel spheres (1-, 1.5-, and 3-mm diameter). The surface properties of these metallic structures 106 were compared against PVC used in hydroponics growing troughs. To achieve comparable surface properties, we experimented with the following fabrication parameters: dip coating vs. Impregnation with iron fillings, solvent used in coating (THF vs. DMF), PVC concentration (6 vs. 8%), multiple coats, and annealing conditions. Early results indicate promising success for dip coating with a double coat of 6% PVC followed by 8% PVC. The 6% PVC coating 108, 110 was found to be optimal for flushness of the exterior surface. The 8% PVC coating 108, 110 was found to be optimal for thickness and durability. The 8% PVC and 6% PVC double coating 108,110 was found to provide both robustness and enhanced smoothness of the exterior. This method provided the most proficient method for maintaining a flush coating 108,110 around the exterior of the metallic structures 106. It is contemplated that other solvent coatings and annealing processes may be utilized. The results of this study indicate that a double coating 108,110 of 8% and 6% PVC solutions exhibited superior smoothness and thickness, ensuring complete coverage and protection against exposure of the steel. The method of increasing the drop length of water showed to be effective in allowing a more uniform and adherent coat on the beads as it allowed sufficient time for solvent dissolution. Leaving beads in the coating solution for the initial application, proved to be effective in enhancing both the durability and flushness of the exterior. This smoothness of the coat was able to be improved even further by tumbling the beads. A skilled artisan may select other ways to improve the smoothness of the coat, within the scope of the present disclosure. It is contemplated the method may be optimized to match the surface roughness of the beads to that of PVC pipe samples.

Advantageously, the system 100 of the present disclosure may automate the analysis of biofilm growth on PVC surfaces for developing safer hydroponic and home water systems.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions, and methods can be made within the scope of the present technology, with substantially similar results.

What is claimed is:

1. A system comprising:
a robot;
a magnet coupled to the robot; and
a metallic structure having a coating that enables microbiome propagation.

2. The system of claim 1, wherein the coating includes polyvinyl chloride (PVC).

3. The system of claim 2, wherein the metallic structure is substantially spherically shaped.

4. The system of claim 2, wherein the PVC coating has a thickness that allows for a magnetic attraction between the magnet and the metallic structure.

5. The system of claim 2, wherein the robot controls a position of the metallic structure.

6. The system of claim 5, wherein the robot controls the position of the metallic structure autonomously.

7. The system of claim 2, wherein the metallic structure includes stainless steel.

8. The system of claim 2, wherein the magnet includes a first magnet and a second magnet, the first magnet controls a position of the metallic structure, and the second magnet controls the selective detachment of the metallic structure from the robot.

9. A hydroponic system utilizing the system of claim 1.

10. A method of manufacturing a system configured to monitor microbiome propagation, the method comprising the steps of:
dissolving PVC powder in a solvent, thus providing a first PVC solution;
exposing a metallic structure to the first PVC solution;
rinsing the metallic structure with deionized water;
removing any residual first PVC solution from the metallic structure; and
coupling the metallic structure to the magnet.

11. The method of claim 10, wherein the solvent is one of Tetrahydrofuran (THF) and Dimethylformamide (DMF).

12. The method of claim 11, further comprising a step of exposing the metallic structure with a second PVC solution after the metallic structure has been coated with the first PVC solution.

13. The method of claim 12, wherein first PVC solution includes around eight percent by weight PVC.

14. The method of claim 13, wherein second PVC solution includes around six percent by weight PVC.

15. The method of claim 10, wherein the step of exposing the metallic structure to the first PVC solution includes a dip coating process.

16. The method of claim 10, wherein the step of exposing a metallic structure to the first PVC solution includes an impregnation process with iron fillings.

17. The method of claim 14, further comprising a step of tumbling the coated metallic structure after the step of exposing the metallic structure with a second PVC solution.

18. The method of claim 10, wherein the step of removing any residual first PVC solution from the metallic structure includes disposing the coated metallic structure in a vacuum oven to remove any residual first PVC solution.
